(19) United States
(12) Patent Application Publication
(10) Pub. No.: US 2025/0289623 A1
(43) Pub. Date: Sep. 18, 2025

(54) SECONDARY PACKAGING CONFIGURED TO HOLD AT LEAST ONE DEVICE FOR ADMINISTERING A DRUG, AND METHOD OF MAKING AND USING SAME

(71) Applicant: CSP Technologies, Inc., Auburn, AL (US)

(72) Inventors: Ethan Ross Perdue, Auburn, AL (US); Kathryn Perko, Auburn, AL (US); Danielle Nevins, Auburn, AL (US); Franklin Lee Lucas, JR., Opelika, AL (US); Donald Lee Huber, Auburn, AL (US)

(21) Appl. No.: 19/100,312

(22) PCT Filed: Jul. 27, 2023
(86) PCT No.: PCT/US2023/028826

§ 371 Date: Jan. 31, 2025

Related U.S. Application Data
(60) Provisional application No. 63/370,045, filed on Aug. 1, 2022.

(51) Int. Cl. B65D 25/10; A61M 11/00; A61M 15/08

(52) U.S. Cl. CPC B65D 25/105; A61M 11/007; A61M 15/08; A61M 2209/06

(57) ABSTRACT

Secondary packaging configured to hold a device for administering a drug can include a body including at least one clip attached to an interior surface thereof. The at least one clip can be configured to surround at least a portion of the device, the clip being configured to prevent inadvertent removal or movement of the device with respect to the body. The secondary packaging can also include a cap pivotably attached to the body. The cap and the body can combine to form a cavity for holding the device when the cap is in a closed position with respect to the body.

### CROSS-REFERENCE TO RELATED APPLICATION

The present application claims priority to U.S. Provisional Application No. 63/370,045, filed Aug. 1, 2022 and titled “PACKAGING CONFIGURED TO HOLD A DEVICE FOR ADMINISTERING MEDICINE OR A DRUG”, which is hereby incorporated by reference in its entirety.

### FIELD

The presently disclosed technology relates generally to secondary packaging configured to hold a device designed to administer contents, such as but not limited to a drug, therein. More particularly, in one embodiment, the presently disclosed technology relates to secondary packaging configured to prevent inadvertent actuation of an inhaler and/or to protect the inhaler during storage, transport, and/or non-use of the inhaler.

### BACKGROUND

Devices for administering or releasing contents, such as one or more drugs, are often stored or transported in secondary packaging. The secondary packaging is designed to protect the device(s) in a compact and economical manner. The secondary packaging can be discarded or reused once the device(s) is/are removed therefrom for use by an end user or healthcare professional, for example.

Inadvertent actuation of and damage to the aforementioned devices within the secondary packaging is not uncommon. Inadvertent actuation wastes valuable contents (e.g., drugs), and can create a mess within and outside of the secondary packaging. Damage to the device(s) can prevent use of the device, which in turn wastes valuable contents and wastes the raw materials used to form the devices.

### SUMMARY

There is a need to prevent inadvertent actuation of the device(s) stored within secondary packaging. There is also a need for simplified and/or different configurations of secondary packaging, which can securely hold and protect one or more of the aforementioned devices in a more compact or economical manner. For example, it would be beneficial to create secondary packaging that is not necessarily child-resistant, but requires or at least benefits from two hands to open. It would also be beneficial to create secondary packaging that is sleek, and convenient to store and transport.

The above and other needs are addressed by the presently disclosed technology.

In one aspect, the presently disclosed technology is directed to secondary packaging configured to hold one or more devices for administering a drug. The secondary packaging can include a body having a base and a sidewall extending upwardly therefrom. The secondary packaging can also include a cap removably attachable to the body. The cap and the body can combine to form a cavity for holding the device when the cap is attached to the body.

In another aspect, the presently disclosed technology is directed to a combination of a nasal dose inhaler, and secondary packaging configured to hold one or more of the nasal dose inhalers.

In yet another aspect, the presently disclosed technology is directed to secondary packaging configured to hold one or more devices for administering a drug. The secondary packaging can include a body and a cap pivotably attached to the body. The cap and the body can combine to form a cavity for holding the device when the cap is attached to the body. The body can include a clip on an interior surface thereof. The clip can be configured to surround at least a portion of the device. The clip can be configured to prevent inadvertent removal or movement of the device with respect to the body.

In still another aspect, the presently disclosed technology is directed to secondary packaging configured to hold a device for administering a drug that can include a body having at least one clip attached to an interior surface thereof. The at least one clip can be configured to surround at least a portion of the device. The clip can be configured to prevent inadvertent removal or movement of the device with respect to the body. The secondary packaging can also include a cap pivotably attached to the body. The cap and the body can combine to form a cavity for holding the device when the cap is in a closed position with respect to the body.

In yet another aspect, the presently disclosed technology is directed to a method of attaching a device for administering a drug to secondary packaging that can include pivoting a cap of the secondary packaging with respect to a body of the secondary packaging about a hinge of the secondary packaging to move the secondary packaging from a closed configuration to an open configuration to expose a cavity. The method can include placing the device for administering the drug into at least one clip attached to an interior surface of the body of the secondary packaging. The at least one clip can be configured to surround at least a portion of the device. The clip can be configured to prevent inadvertent removal or movement of the device with respect to the body.

Optionally, the device is snap-fit into the clip.

### BRIEF DESCRIPTION OF THE DRAWINGS

The following detailed description of the presently disclosed technology, will be better understood when read in conjunction with the appended drawings, wherein like numerals designate like elements throughout. For the purpose of illustrating the presently disclosed technology, there are shown in the drawings various illustrative embodiments. It should be understood, however, that the presently disclosed technology is not limited to the precise arrangements and instrumentalities shown. In the drawings:

FIG. 1 is a perspective view of secondary packaging according to an optional aspect of the presently disclosed technology. wherein the secondary packaging is shown in a first or closed position;

FIG. 2 is a front elevation view of the secondary packaging shown in FIG. 1;

FIG. 3 is a side elevation view of the secondary packaging shown in FIG. 1;

FIG. 4 is a cross-sectional side elevation view of the secondary packaging taken along line A-A of FIG. 1;

FIG. 4a is a perspective view of an optional device configured to be held by the secondary packaging shown in FIG. 1;

FIG. 5 is a top plan view of the secondary packaging shown in FIG. 1;

FIG. 6 is a partially exploded perspective view of the secondary packaging shown in FIG. 1, wherein the secondary packaging is in a second or open position and an optional device for administering a drug according to FIG. 4a is shown therein;

FIG. 7 is another partially exploded perspective view of the secondary packaging shown in FIG. 1, wherein the secondary packaging is in the open position and the device of FIG. 4a is shown therein;

FIG. 8 is yet another partially exploded perspective view of the secondary packaging shown in FIG. 1, wherein the secondary packaging is in the open position and the device of FIG. 4a is shown therein;

FIG. 9 is a perspective view of secondary packaging according to another optional aspect of the presently disclosed technology, wherein the secondary packaging is shown in a first or closed position;

FIG. 10 is a top plan view of the secondary packaging shown in FIG. 9;

FIG. 11 is a front elevation view of the secondary packaging shown in FIG. 9;

FIG. 12 is a right side elevation view of the secondary packaging shown in FIG. 9;

FIG. 13 is a top plan view of the secondary packaging shown in FIG. 9, wherein the secondary packaging is shown in a second or open position and a device for administering a drug is shown therein;

FIG. 14 is another perspective view of the secondary packaging shown in FIG. 9, wherein the secondary packaging is shown in the open position and the device is shown therein;

FIG. 15 is another perspective view of the secondary packaging shown in FIG. 9, wherein the device is removed for clarity;

FIG. 16 is a perspective view of secondary packaging according to a modified version of that shown in FIG. 9, wherein the secondary packaging is shown in a closed position;

FIG. 17 is a top plan view of the secondary packaging shown in FIG. 16;

FIG. 18 is a front elevation view of the secondary packaging shown in FIG. 16;

FIG. 19 is a right side elevation view of the secondary packaging shown in FIG. 16; and

FIG. 20 is a top plan view of the secondary packaging shown in FIG. 16, wherein the secondary packaging is shown in a second or open position and two devices for administering a drug are shown therein.

### DETAILED DESCRIPTION

While systems, devices and methods are described herein by way of examples and embodiments, those skilled in the art recognize that the presently disclosed technology is not limited to the embodiments or drawings described. Rather, the presently disclosed technology covers all modifications, equivalents and alternatives falling within the spirit and scope of the appended claims. Features of any one embodiment disclosed herein can be omitted or incorporated into another embodiment.

Any headings used herein are for organizational purposes only and are not meant to limit the scope of the description or the claims. As used herein, the word “may” is used in a permissive sense (i.e., meaning having the potential to) rather than the mandatory sense (i.e., meaning must). Unless specifically set forth herein, the terms “a,” “an” and “the” are not limited to one element but instead should be read as meaning “at least one.” The terminology includes the words noted above, derivatives thereof and words of similar import.

As used herein, “and/or” means that either or both of the items separated by such terminology are involved. For example, the phrase “A and/or B” would mean A alone, B alone, or both A and B.

As used herein, “generally” means “in a general manner” relevant to the term being modified as would be understood by one of ordinary skill in the art.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In one optional embodiment, the secondary packaging described herein is not moisture tight.

In another optional embodiment, the secondary packaging described herein is moisture tight and resealable. Generally speaking, as used herein, the term “moisture tight” is defined as having a moisture ingress (after three days) of less than 1500 μg of water, in another embodiment, less than 500 μg of water, in a further embodiment, less than 300 μg of water, in yet another embodiment, less than 150 μg of water, as determined by the following test method: (a) place one gram plus or minus 0.25 grams of molecular sieve in the container and record the weight; (b) fully close the container; (c) place the closed container in an environmental chamber at conditions of 80% relative humidity and 72° F.; (d) after one day, weigh the container containing the molecular sieve; (e) after four days, weigh the container containing the molecular sieve; and (f) subtract the first day sample from the fourth day sample to calculate the moisture ingress of the container in units of micrograms of water. A preferred rate of ingress of moisture into a moisture tight sealed container produced according to an aspect of the disclosed concept is in the range of about 200-300 μg/day of water or less. A “moisture tight” seal therefore is a sealing engagement that alone, or in combination with additional sealing engagements, help to render a container “moisture tight” per the above definition.

As used herein, the term “resealable” means the lid of the container can be opened or reopened and closed or reclosed many times (e.g., more than 10 times) and still retain its moisture-tight properties.

As used herein, the term “secondary” in the phrase “secondary packaging” means that the packaging does not directly contain or hold contents consumed by the user, but instead holds the packaging that directly contains or holds the contents consumed by the user.

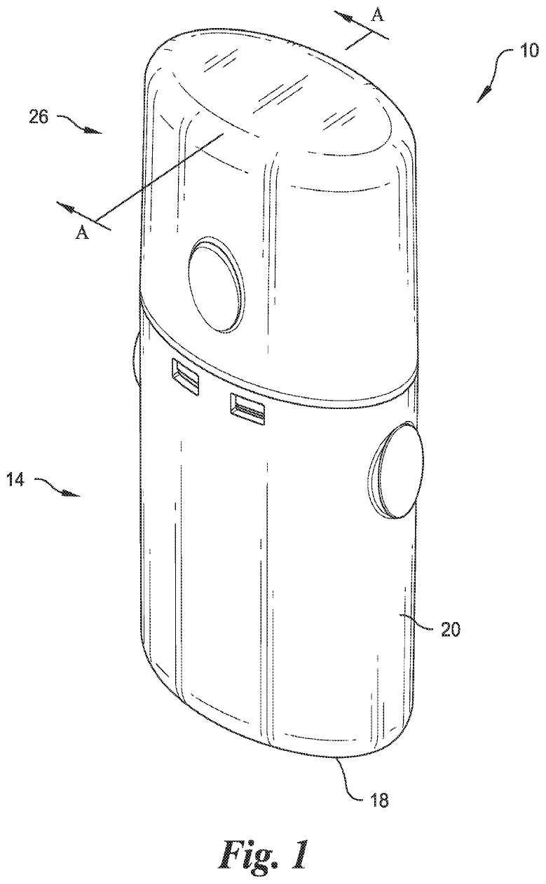

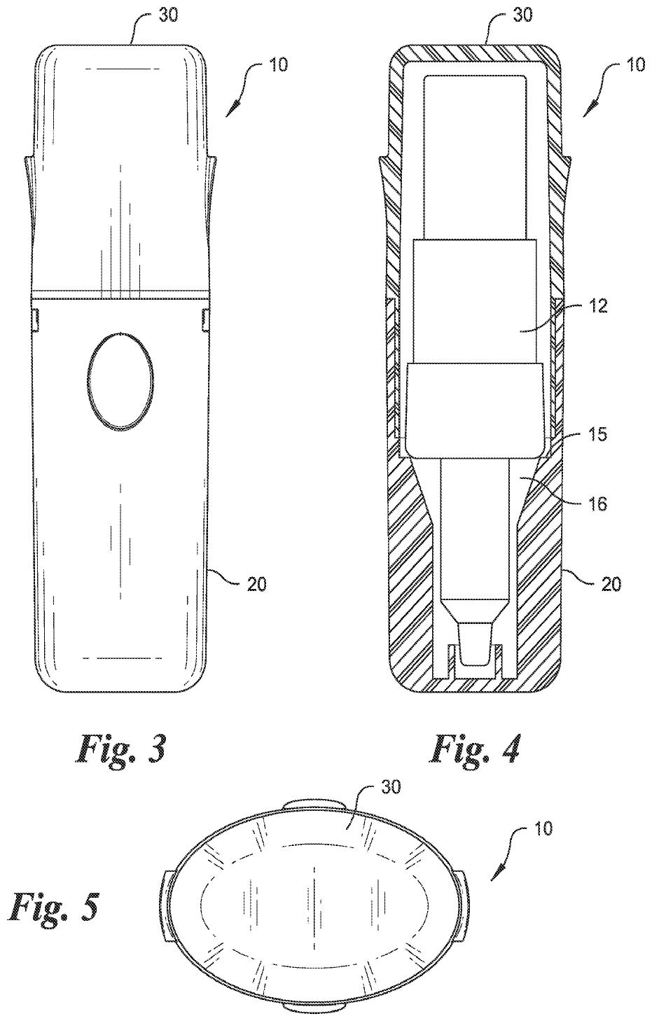

Referring now in detail to the various figures, wherein like reference numerals refer to like parts throughout, FIGS. 1-8 show a combination of secondary packaging, generally designed 10, and at least one device, generally designated 12. Each device 12 can be configured for storing, releasing, and/or administering contents therein, such as but not limited to a drug. The secondary packaging 10 can be configured to hold or container one or more of the devices 12 therein, and prevent inadvertent actuation, e.g., through axial compression, of the device 12.

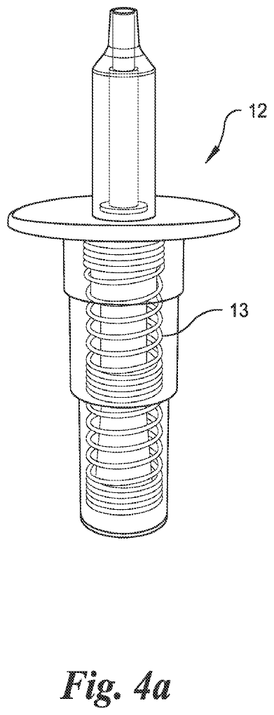

Optionally, the device 12 is an inhaler, such as but not limited to nasal inhalers, metered-dose inhalers (MDIs), dry powder inhalers (DPIs), and soft mist inhalers (SMIs). The inhaler can be a unidose (UDS) or bidose (BDS) inhaler. The device 12 can optionally include a spring 13 (see FIG. 4a) to facilitate movement (e.g., expansion and/or contraction) of the device 12 from an activated position in which at least some of the contents of the device 12 are released or administered, to a non-activated position in which contents of the device 12 are prevented from exiting. However, the device 12 is not limited to being an inhaler, as the device 12 can be any structure configured to store, release, and/or administer contents held therein, such as an eyedropper or a pepper sprayer.

Optionally, an active pharmaceutical ingredient (API) stored within and/or delivered by the device 12 can be one or more of Naloxone, Ketamine, Midazolam, Fentanyl, Lorazepam, Glucagon, Nalmefene, Epinephrine, Apomorphine, Cetrorelix, Loxapine, Eletriptan, Ketorolac, and/or Olanzapine.

The device 12 can optionally be employed to store and/or deliver insulin and/or epinephrine and/or another medicament to treat or reduce the symptoms of diabetes or asthma, for example.

In one optional embodiment, the device 12 can be pre-filled and provide a medication in dosage form. In particular, the device 12 can be a dispersion device that delivers a medication in a dispersed form, such as in metered aerosol or spray devices. The device 12 can be pre-filled with the medication(s) in a finished dosage form. Embodiments include devices such as referenced in 68 Fed. Reg. 36,675, 36,676 and 36,680 (Jun. 18, 2003). Exemplary embodiments include intranasal aerosol and spray devices.

The secondary packaging 10 is particularly well suited for use with devices 12 that are used for administration of powder medications, such as intranasal delivery powders. The medications can be any that can be delivered by a dispersion device. The medication(s) can be associated with one or more other ingredients, as referenced, for example, at 21 C.F.R. § 314.3.

In an optional embodiment, the medication stored within the device 12 is a powder medicament, such as glucagon. The device 12 can be configured for the delivery of glucagon or another medicament in a simple, one-use nasal powder. This provides significant advantages over complicated systems that involve mixing liquid and powder together and then injecting the solution. The use of the device 12 by a patient has been determined to bring blood sugar levels close to normal within 30 minutes of taking the powdered glucagon.

In one optional embodiment, the nasal powder can be administered with a single-use, one-step dispensing device. The tip of the device 12 can be inserted in one nostril, and the dose can be delivered by simply depressing an actuator connected to a piston that discharges the powder into the nostril. No cooperative measure is required from the patient, as absorption takes place through the nasal mucosa. The glucagon formulation is provided in the device 12, resulting in a highly effective protection for the viability of both the glucagon and its delivery device.

Further details regarding the device 12 or similar devices that may be used according to optional aspects of the presently disclosed technology are disclosed in U.S. Pat. No. 10,894,133, which is incorporated by reference herein in its entirety.

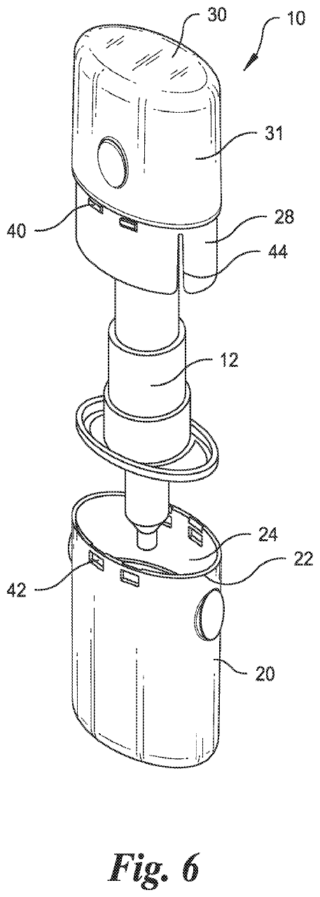

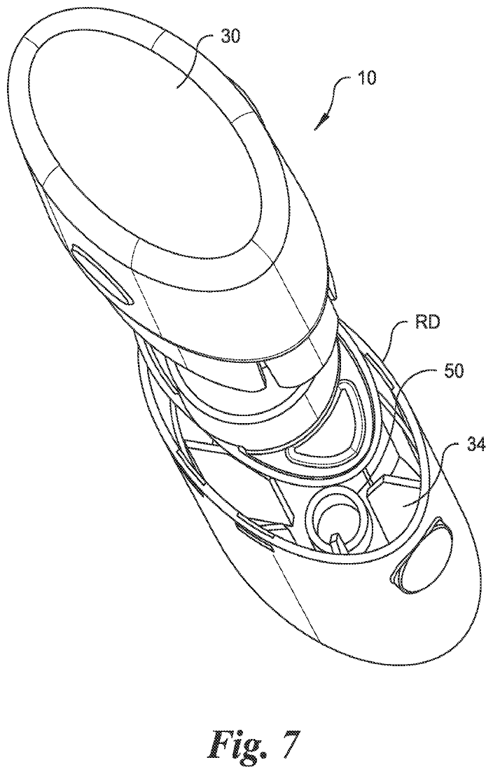

The secondary packaging 10 can include a body 14 having a base 18 and a sidewall 20 extending upwardly from the base 18. An end of the sidewall 20 opposite the base 18 can include a rim 22 (see FIGS. 6 and 7) that surrounds and/or defines an opening 24 leading to the cavity 16. Optionally, the opening 24 is oval or circular.

The secondary packaging 10 can also include a cover or cap 26 removably attachable to and/or separable from (e.g., spaced-apart from) the body 14. The cap 26 can include a top wall 30 and a sidewall 31 extending downwardly therefrom. The cap 26 can include an annular skirt 28 extending downwardly (directly or indirectly) from the sidewall 31 and/or a top portion of the cap 26. In the present optional embodiment, the top wall 30 of the cap 26 is at least partially or completely planar. Alternatively, the top wall 30 can be partially or completely arcuate.

In one embodiment, the cap 26, the body 14, and/or the other features of the secondary packaging 10 are optionally made primarily or exclusively from one or more injection moldable thermoplastic materials, including, for example, a polyolefin such as polypropylene (PP) or polyethylene. It can be beneficial to make the entire secondary packaging 10 and the components thereof out of a single material to make recycling easier.

In the present optional embodiment, the cap 26 is movable with respect to and/or separable from the body 14 between a first or closed position (e.g., FIGS. 1-5) in which the cap 26 and the body 14 can optionally form a seal, such as but not limited to a moisture-tight seal, and a second or open position (e.g., FIGS. 6-8) in which the opening 24 of the body 14 is exposed. Optionally, at least a portion of the skirt 28 or the entirety of the skirt 28 is configured to fit within at least a portion of the sidewall 20 of the body 14 when the cap 26 is attached to the body 14.

In the closed position, the cap 26 and the body 14 can combine to form a cavity 16 (see FIG. 4) for holding, protecting, and/or surrounding the device 12 when the cap 26 is attached to the body 14. The cavity 16 can be configured to receive the entire device 12.

In one optional embodiment, the cap 26 is completely separable from the body 14 in the open position, such that the packaging 10 is formed of two distinct or separate pieces. In an alternative optional embodiment, a hinge can connect the cap 26 to the body 14. The cap 26 can optionally be resealable with respect to the body 14.

In the present optional embodiment. the body 14 and the cap 26 can combine to form a cylinder or bottle, such that the sidewall 20 of the body 14 and the sidewall 31 of the cap 26 can be at least partially arcuate or round. However, the disclosed concept may include or be other shapes, e.g., rectangular or cuboid, and thus have more than a single continuous (e.g., round) sidewall.

For certain uses, a child-resistant cap or packaging may be desired, but may not be necessary for all applications. Thus, child-resistant and non-child-resistant caps or packages are contemplated. If a child-resistant feature is provided, the child-resistant feature optionally requires that force in more than one single direction is applied to the cap 26 to remove the cap 26 from the body 14.

Optionally, when the device 12 is within the cavity 16 (e.g., when the cap 26 is attached to the body 14), at least a portion of the body 14, the cap 26, and/or the secondary packaging 10 is configured to prevent and/or includes means for preventing movement of the device 12 that would otherwise release or administer the drug. For example, an interior surface of the body 14 can include a ledge 15 (see FIG. 4a) that is configured to contact a portion of the device 12 and prevent the device 12 from moving from the non-activated position to the activated position when the cap 26 is attached to the body 14.

As shown in FIG. 7, the means for preventing movement of the device 12 that would otherwise release or administer the drug can include, or the packaging 10 can include, at least one or two or more spaced-apart fins or ribs 34 extending inwardly into the cavity 16 from an interior surface of the body 14. In one optional embodiment, each fin 34 can extend inwardly and/or upwardly from an interior surface of the base 18 of the body 14. A top surface of one or more of the fins 34 can form the ledge 15.

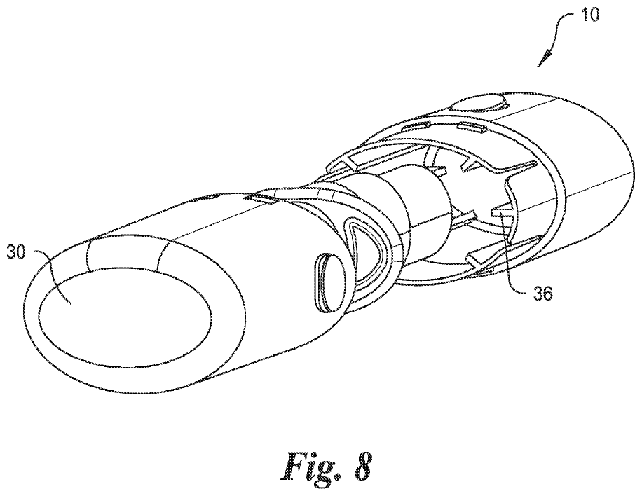

Alternatively or additionally. as shown in FIG. 8, the means for preventing movement of the device 12 that would otherwise release or administer the drug can include, or the packaging 10 can include, at least one or two or more spaced-apart fins or ribs 36 extending inwardly into the cavity 16 from an interior surface of the cap 26. In one optional embodiment, each fin 36 can extend inwardly and/or upwardly from an interior surface of the top wall of the cap 26.

Optionally, when the cap 26 is in the closed position and the device 12 is within the cavity 16, the fins 34, 36 can be positioned on either side of an upper and lower end of the device 12 to help maintain the upper end of the device 12 in a particular or desirable lateral and longitudinal position. Each fin 34, 36 can be configured to prevent the device 12 from being reconfigured from the non-activated position to the activated position. Each fin 34, 36 can be of sufficient length and/or width to contact (or at least come close to contacting) a portion of the device 12 when the device 12 is positioned into the cavity 16 and the cap 26 is in the closed position so as to block or prevent the device 12 from moving, even slightly, from the non-activated position to the activated position (i.e., prevent the device from being compressed axially).

Referring to FIGS. 6 and 8, the skirt 28 of the cap 26 can optionally include at least one or two or more spaced-apart projections 40 extending at least slightly radially outwardly therefrom. Each projection 40 can be located closer to the end of the skirt 28 near the sidewall 31 of the cap 26 than a free end of the skirt 28. Optionally, two spaced-apart projections 40 can be linearly arranged across a width of the skirt 28.

In one optional embodiment, the sidewall 20 of the body 14 can include at least one or two or more spaced-apart openings or grooves 42 extending therein or therethrough. Each opening 42 can be configured to receive at least a portion of one projection 40 when the cap 26 is in the closed position with respect to the body 14. Engagement or disengagement of each projection 40 and opening 42 combination can provide a tactile indication to the user that the packaging 10 has been closed (e.g., by feeling or hearing a snap or click) or opened, respectively. Of course, each projection 40 and opening/groove 42 can be reversed in location, such that each projection 40 is located on the body 14.

As shown in FIG. 7, the thickness of the sidewall 20 of the body 14 proximate the rim 26 and each opening or groove 42 of the body 14 can have a reduced dimension (e.g., in the radial direction), generally designated RD, such that the reduced thickness of the sidewall 20 at each of these locations allows one of the projections 40 to more easily enter and be removed from the respective opening or groove 42. Each reduced thickness area can have a lateral width identical or similar to that of the opening or groove 42 to which it corresponds.

As shown in FIGS. 6-8, the skirt 28 can optionally include at least one or a plurality of spaced-apart cut-outs 44. Each cut-out 44 can be wider at the free end of the skirt 28 and narrow or taper as the cut-out moves toward the opposite end of the skirt 28. Each cut-out 44 provides flexibility to the skirt 28, which can help facilitate moving the cap 26 from the opened to the closed position and prevent or reduce the likelihood of undesirable warping or deflection of the components.

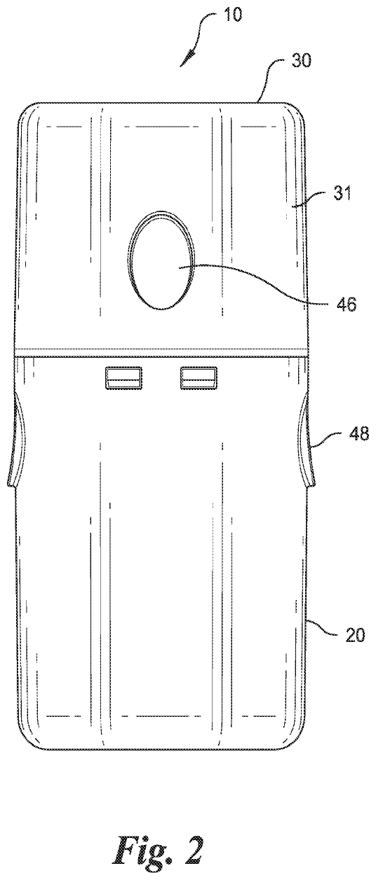

In one optional embodiment, as shown in FIG. 2, the cap 26 can include one or at least two opposing cap thumb tabs 46 and the body 14 can include one at least two opposing body thumb tabs 48. In an optional embodiment wherein the package 10 has an oval configuration when viewed from below or above. the cap thumb tabs 46 can be aligned along the major axis, while the body thumb tabs 48 can be aligned along the minor axis, which is perpendicular to the major axis.

Optionally, each thumb tab 46, 48 is fixed or stationary with respect to the respective sidewall 20, 31. Alternatively, each thumb tab 46, 48 can be movable with respect to the respective sidewall 20, 31. In the latter example, simultaneously engaging at least two of the opposing cap or body thumb tabs 46, 48 causes the projection 40 to disengage from the respective opening or groove 42.

One or both of the thumb tabs 46, 48 or set of thumb tabs 46, 48 can be configured to be engaged and/or depressed by a user to facilitate separation of the cap 26 from the body 14 and/or attachment of the cap 26 to the body 14. In particular, laterally inward movement of one or both of the thumb tabs 46, 48 or set of thumb tabs 46, 48, such as by a user's fingers, can help facilitate engagement or separation of the projection(s) 40 and the opening(s) 42.

Referring to FIG. 7, a ring 50 can optionally extend inwardly or upwardly from an interior surface of the base 18 of the body 14. The ring 50 can be configured to receive at least a portion of the device 12 when the device 12 is placed in the body 14. The ring 50 can help prevent inadvertent or undesirable movement of the device 12 with respect to the body 14, and/or reduce the likelihood of damage to the device 12 inside the secondary packaging 10.

Optionally, the secondary packaging 10, for example the cap 26 and/or the body 14 thereof, can include an active polymer component that is affixed to a portion thereof or integral therewith. For example, the active polymer component can be part of the molding process of the body 14 and/or the cap 26, such that the end user cannot visually identify the active polymer component from the body 14 and/or the cap 26.

In another optional embodiment, the secondary packaging 10 is devoid of an active polymer component.

Optionally, the body 14 and/or the cap 26 can be formed of a two-shot or three-shot injection molding process, as described in detail below.

The active polymer component can include a base polymer entrained with one or more active agents and thus may be referred to herein as a polymer entrained with an active agent or entrained polymer. The active agent in the active polymer component may include an absorbing material, a releasing material and/or an activation material. Optionally, the active polymer component is a three-phase desiccant entrained polymer. The active polymer component can be provided in different shapes, volumes and/or configurations. In one exemplary embodiment, the active polymer component is in the form of a solid plug or generally planar member, extending into an interior space of the cap 26.

In one embodiment, active polymer component is a desiccant entrained polymer that is a unitary component made of a single piece of material. An entrained polymer, whether entrained with desiccant or another active agent, may include a base polymer (for structure), a desiccant (or other active agent) and optionally a channeling agent. These types of active entrained polymers and methods of making and using the same are disclosed. e.g., in Applicant's U.S. Pat. Nos. 5,911,937, 6,214,255, 6,130,263, 6,080,350, 6,174,952, 6,124,006 and 6,221,446, and U.S. Pat. Pub. No. 2016/0039955, which are each incorporated by reference in their entirety herein. Optionally, the entrained polymer may be in the form of a film that is loose or optionally heat-staked (e.g., via a heat seal to a surface).

Alternatively, the desiccant may include loose desiccant beads or a sachet containing the same. While the exemplary embodiments herein reflect active polymer component being attached, for example, to the cap 26 or the body 14, it is contemplated that an active agent can be located at other locations and/or positions, such as on a sidewall of the body.

In the embodiment where each active member contains a desiccant, moisture absorption is desired. However, where moisture absorption is not desired, the active member can include alternative active agents. For example, in another embodiment, the active member contains a material selected from the group consisting of activated carbon, carbon black, ketjenblack, and diamond powder. In a further embodiment, an active agent including one or more layers of the active member contains a material such as absorption microspheres, BaTiO3, SrTiO3, SiO2, Al2O3, ZnO, TiO2, MnO, CuO, Sb2O3, silica, calcium oxide and ion exchange resins. In yet another embodiment, the absorbing agent containing layer of the active member contains two or more types of absorbing agents. The suitable absorbing agent is chosen to achieve absorption of the desired vapor or gas for the desired end use (e.g., absorption of moisture, oxygen, carbon dioxide, nitrogen or other undesired gases or vapors).

The active member (whether desiccant, oxygen scavenger, a releasing material or ingredient, etc., or combination thereof) is capable of acting on, interacting or reacting with a selected material (e.g., moisture or oxygen). Examples of such actions or interactions may include absorption. adsorption (sorption, generally) or release of the selected material. Each active member can be extruded or molded, for example. Optionally, the active member can be formed in a desired shape or pattern (e.g., on a backing) via an in-line melt adhesion thermal bonding process.

The active member can include an “active ingredient” in a base material. The active ingredient(s) (i) can be immiscible with the base material (e.g., polymer) and when mixed and heated with the base polymer and a channeling agent, will not melt, i.e., has a melting point that is higher than the melting point for either the base polymer or the channeling agent, and/or (ii) acts on, interacts or reacts with a selected material. The term “active ingredient” may include but is not limited to materials that absorb, adsorb or release the selected material(s). Active ingredients, according to the presently disclosed technology, may be in the form of particles such as minerals (e.g., molecular sieve or silica gel, in the case of desiccants), but the presently disclosed technology should not be viewed as limited only to particulate active agents. For example, in some embodiments, an oxygen scavenging formulation may be made from a resin which acts as, or as a component of, the active agent.

As used herein, the term “base material” is a component (preferably a polymer) of an entrained active material, other than the active agent, that provides structure for the entrained material.

As used herein, the term “base polymer” is a polymer optionally having a gas transmission rate of a selected material that is substantially lower than, lower than or substantially equivalent to, that of the channeling agent. By way of example, such a transmission rate would be a water vapor transmission rate in embodiments where the selected material is moisture, and the active ingredient is a water absorbing desiccant. The primary function of the base polymer is to provide structure for the entrained polymer. Suitable base polymers may include thermoplastic polymers, e.g., polyolefins such as polypropylene and polyethylene, polyisoprene, polybutadiene, polybutene, polysiloxane, polycarbonates, polyamides, ethylene-vinyl acetate copolymers, ethylene-methacrylate copolymer, poly(vinyl chloride), polystyrene, polyesters, polyanhydrides, polyacrylonitrile, polysulfones, polyacrylic ester, acrylic, polyurethane and polyacetal, or copolymers or mixtures thereof.

Referring to such a comparison of the base polymer and channeling agent water vapor transmission rate, in one embodiment, the channeling agent has a water vapor transmission rate of at least two times that of the base polymer. In another embodiment, the channeling agent has a water vapor transmission rate of at least five times that of the base polymer. In another embodiment, the channeling agent has a water vapor transmission rate of at least ten times that of the base polymer. In still another embodiment, the channeling agent has a water vapor transmission rate of at least twenty times that of the base polymer. In still another embodiment, the channeling agent has a water vapor transmission rate of at least fifty times that of the base polymer. In still another embodiment, the channeling agent has a water vapor transmission rate of at least one hundred times that of the base polymer.

As used herein, the term “channeling agent” or “channeling agents” is defined as a material that is immiscible with the base polymer and has an affinity to transport a gas phase substance at a faster rate than the base polymer. Optionally, a channeling agent is capable of forming channels through the entrained polymer when formed by mixing the channeling agent with the base polymer. Optionally, such channels are capable of transmitting a selected material through the entrained polymer at a faster rate than in solely the base polymer.

As used herein, the term “channels” or “interconnecting channels” is defined as passages formed of the channeling agent that penetrate through the base polymer and may be interconnected with each other.

As used herein, the term “entrained polymer” is defined as a monolithic material formed of at least a base polymer with an active agent and optionally also a channeling agent entrained or distributed throughout. An entrained polymer thus includes two-phase polymers and three phase polymers. A “mineral loaded polymer” is a type of entrained polymer, wherein the active agent is in the form of minerals, e.g., mineral particles such as molecular sieve or silica gel. The term “entrained material” is used herein to connote a monolithic material comprising an active agent entrained in a base material wherein the base material may or may not be polymeric.

As used herein, the term “monolithic,” “monolithic structure” or “monolithic composition” is defined as a composition or material that does not consist of two or more discrete macroscopic layers or portions. Accordingly, a “monolithic composition” does not include a multi-layer composite, however a monolithic composition could make up a layer of such a composite.

As used herein, the term “phase” is defined as a portion or component of a monolithic structure or composition that is uniformly distributed throughout, to give the structure or composition its monolithic characteristics.

As used herein, the term “selected material” is defined as a material that is acted upon, by, or interacts or reacts with an active agent and is capable of being transmitted through the channels of an entrained polymer. For example, in embodiments in which a desiccant is used as an active agent, the selected material may be moisture or a gas that can be absorbed by the desiccant. In embodiments in which a releasing material is used as an active agent, the selected material may be an agent released by the releasing material, such as moisture, fragrance, or an antimicrobial agent (e.g., chlorine dioxide). In embodiments in which an adsorbing material is used as an active ingredient, the selected material may be certain volatile organic compounds and the adsorbing material may be activated carbon.

As used herein, the term “three phase” is defined as a monolithic composition or structure including three or more phases. An example of a three-phase composition according to the presently disclosed technology would be an entrained polymer formed of a base polymer, active agent, and channeling agent. Optionally, a three-phase composition or structure may include an additional phase, e.g., a colorant.

Entrained polymers may be two phase formulations (i.e., comprising a base polymer and active ingredient, without a channeling agent) or three phase formulations (i.e., comprising a base polymer, active agent and channeling agent). Entrained polymers are described, for example, in U.S. Pat. Nos. 5,911,937, 6,080,350, 6,124,006, 6,130.263, 6,194,079, 6.214,255, 6,486,231, 7,005,459, and U.S. Pat. Pub. No. 2016/0039955, as referenced above.

An entrained material or polymer includes a base material (e.g., polymer) for providing structure, optionally a channeling agent and an active agent. The channeling agent forms microscopic interconnecting channels through the entrained polymer. At least some of the active ingredient is contained within these channels, such that the channels communicate between the active ingredient and the exterior of the entrained polymer via microscopic channel openings formed at outer surfaces of the entrained polymer. The active ingredient can be, for example, any one of a variety of absorbing, adsorbing or releasing materials, as described in further detail below. While a channeling ingredient is preferred, the presently disclosed technology broadly includes entrained materials that optionally do not include channeling agents, e.g., two phase polymers.

In any embodiment, suitable channeling agents may include a polyglycol such as polyethylene glycol (PEG), ethylene-vinyl alcohol (EVOH), polyvinyl alcohol (PVOH), glycerin polyamine, polyurethane and polycarboxylic acid including polyacrylic acid or polymethacrylic acid. Alternatively, the channeling agent can be, for example, a water insoluble polymer, such as a propylene oxide polymerisate-monobutyl ether, such as Polyglykol B01/240, produced by CLARIANT. In other embodiments, the channeling agent could be a propylene oxide polymerisate monobutyl ether, such as Polyglykol B01/20, produced by CLARIANT, propylene oxide polymerisate, such as Polyglykol D01/240, produced by CLARIANT, ethylene vinyl acetate, nylon 6, nylon 66, or any combination of the foregoing.

Suitable active ingredients according to the presently disclosed technology include absorbing materials, such as desiccating compounds. If the active agent is a desiccant, any suitable desiccant for a given application may be used. Typically, physical absorption desiccants are preferred for many applications. These may include molecular sieves, silica gels, clays and starches. Alternatively, the desiccant may be a chemical compound that forms crystals containing water or compounds which react with water to form new compounds.

Optionally, in any embodiment, the active agent may be an oxygen scavenger, e.g., an oxygen scavenging resin formulation.

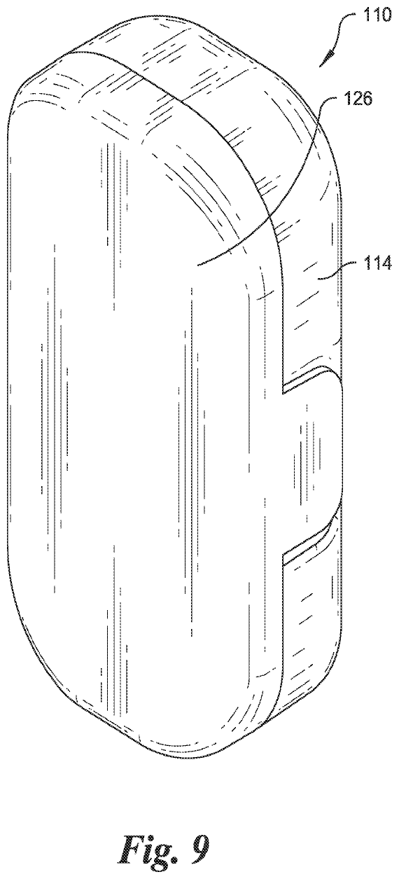

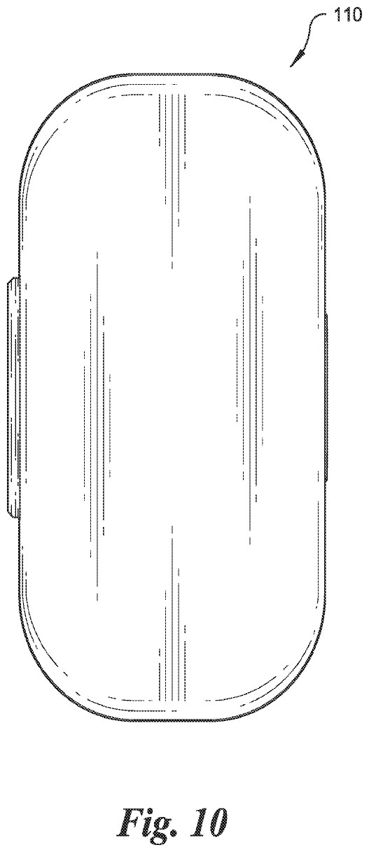

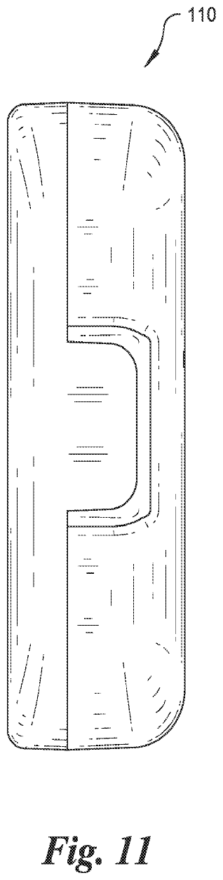

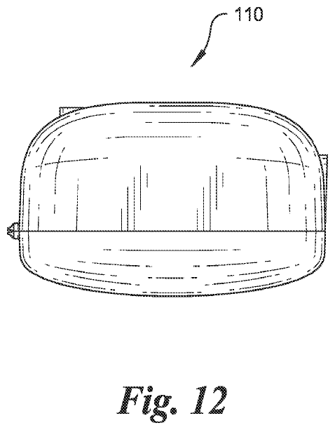

FIGS. 9-15 show another optional embodiment of the combination of secondary packaging 110 and device 112 of the presently disclosed technology. The combination shown in FIGS. 9-15 has certain components or aspects that are similar or identical to the combination shown in FIGS. 1-8 and discussed above. As such, similar or identical structure between the embodiment of FIGS. 1-8 and the embodiment of FIGS. 9-15 is distinguished in FIGS. 9-15 by a reference number with a magnitude one hundred (100) greater than that of FIGS. 1-8. Description of certain similarities between the embodiment of FIGS. 1-8 and the embodiment of FIGS. 9-15 may be omitted herein for convenience and brevity only.

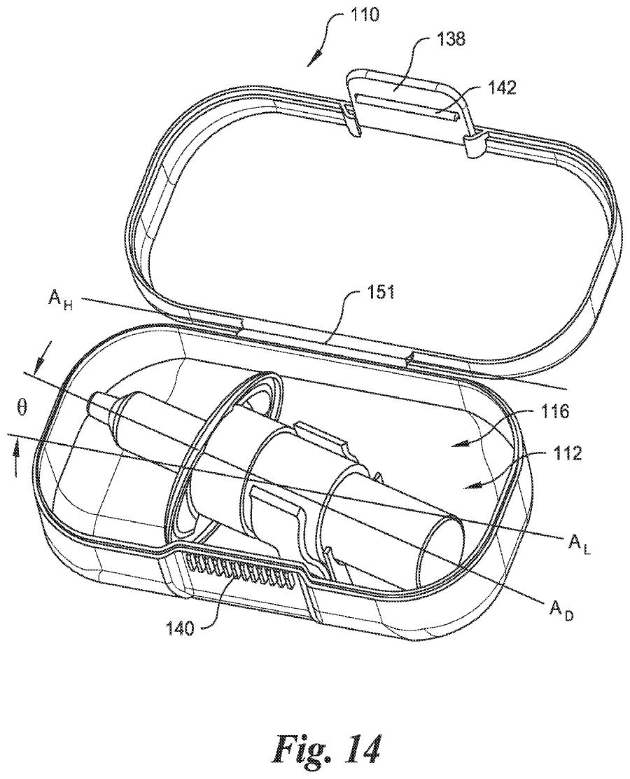

In contrast to the earlier embodiment, the secondary packaging 110 of the presently disclosed embodiment is a one piece or unitary design. In particular, the cap 126 is pivotally attached to the body 114 optionally by at least one hinge 151. When the cap 126 is in the closed position, the cap 126 and the body 114 combine to form the cavity 116 (see FIG. 14) to hold and/or surround the device 112. The secondary packaging 110 can optionally have a clam shell configuration.

One distinguishing feature of the secondary packaging 110 is the at least one clip or attachment mechanism 152 located or fixed to an interior surface thereof. The clip 152 can be configured to surround and/or contact at least a portion of the device 112, but not necessarily an entirety of the device 112. Each clip 152 can be configured to prevent or at least reduce the likelihood of inadvertent removal of the device 112 from the secondary packaging 110.

Optionally, the secondary packaging 110 and/or the clip 152 is configured so that the device 112 can move at least slightly with respect to the clip 152, such as along a longitudinal axis AL (see FIG. 14) of the device 112. In one optional embodiment, such at least slight movement of the device 112 can help prevent damage to the device 112 inside the secondary packaging 110, such as when the secondary packaging 110 is dropped.

In another optional embodiment, the device 112 is fixed and/or not movable along its longitudinal axis A_{L} when the device 112 is in and/or attached to the clip 152.

In the optional embodiment shown, the secondary packaging 110 includes a single clip 152 configured to hold, directly grasp, and/or surround at least a portion of a single device 112. However, the clip 152 can be designed to hold more than one device 112 in a larger version of the secondary packaging 110, or the secondary packaging 110 can include two or more spaced-apart clips 152 each sized to hold one device 112. The clip 152 can also be configured to prevent inadvertent removal or movement of the device 112 with respect to the body 114.

Optionally, the clip 152 is removably attached or permanently fixed to the interior surface of the body 114, such as but not limited to by adhesive, a mechanical fastener (e.g., a pin or screw), or a press-fit or snap-fit engagement. The clip 152 can be positioned or fixed to the body 114 at or near the mid-point of the body 114 along a longitudinal axis A_{L} (see FIG. 14) of the secondary packaging 110. Optionally, the longitudinal axis A_{L} of the secondary packaging 110 extends parallel to an axis defined by the hinge 151 (sometimes referred to herein as the hinge axis A_{H}, as shown in FIG. 14).

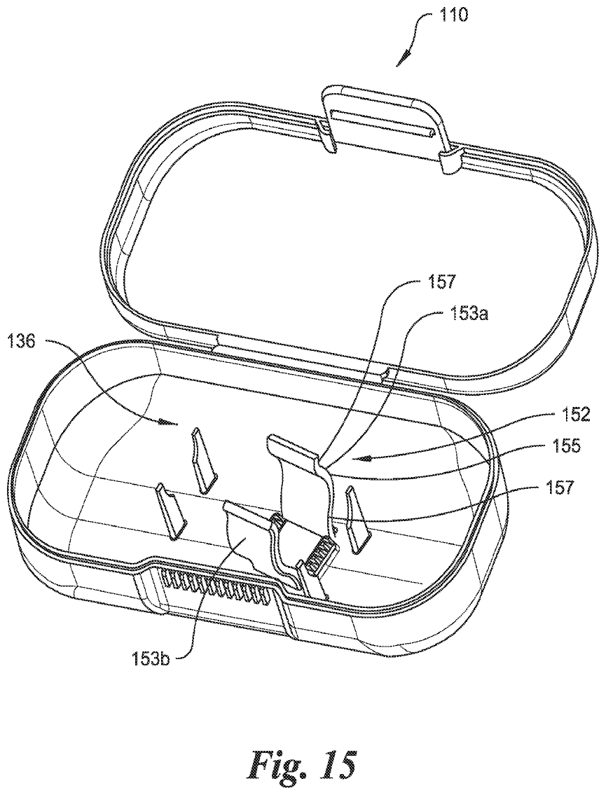

The clip 152 can optionally include two opposing or spaced-apart and arcuate arms 153a, 153b. Each arm 153a. 153b can extend outwardly or away from the interior surface of the body 114 in a non-linear manner. Each arm 153a, 153b can be resilient or biased inwardly (e.g., to the position shown in FIGS. 14 and 15). such that the device 112 can be snap-fit into the clip 152. Optionally, each arm 153a, 153b can be configured to move or flex at least slightly outwardly away from the device 112 to allow the device 112 to be removed from the secondary packaging 110. Thus, each arm 153a. 153b can act as a spring to maintain the device 112 in a desired position or location.

Optionally, each arm 153a, 153b can have a uniform thickness from a base to a free end thereof, as shown in FIG. 15. In an alternative optional embodiment, each arm 153a, 153b can include a portion of reduced thickness or width. In such an embodiment, each arm 153a, 153b can at least slightly flex at the thin area or portion and toward the body 114 if or when the secondary packaging 110 experiences a sudden force, such as upon dropping the secondary packaging 110.

Referring to FIG. 15, in one optional embodiment, each arm 153a, 153b can include at least one first portion 155 and at least one or two spaced-apart second portions 157. Optionally, the first portion 155 can separate the two second portions 157. Each first portion 155 can be concave with respect to the longitudinal axis A_{D} of the device 112 and each second portion 157 can be convex with respect to the longitudinal axis A_{D} of the device 112. Such a configuration allows the clip 152 to easily accommodate and/or receive the device 112, while securely holding the device 112 once the device 112 is pushed sufficiently down into the clip 152.

Optionally, the entire clip 152 or one or more portions thereof can be formed of a metal, while a remainder of the secondary packaging 110 can be formed of a polymeric material. Optionally, the clip 152 and the remainder of the secondary packaging 110 can be formed of a polymeric material.

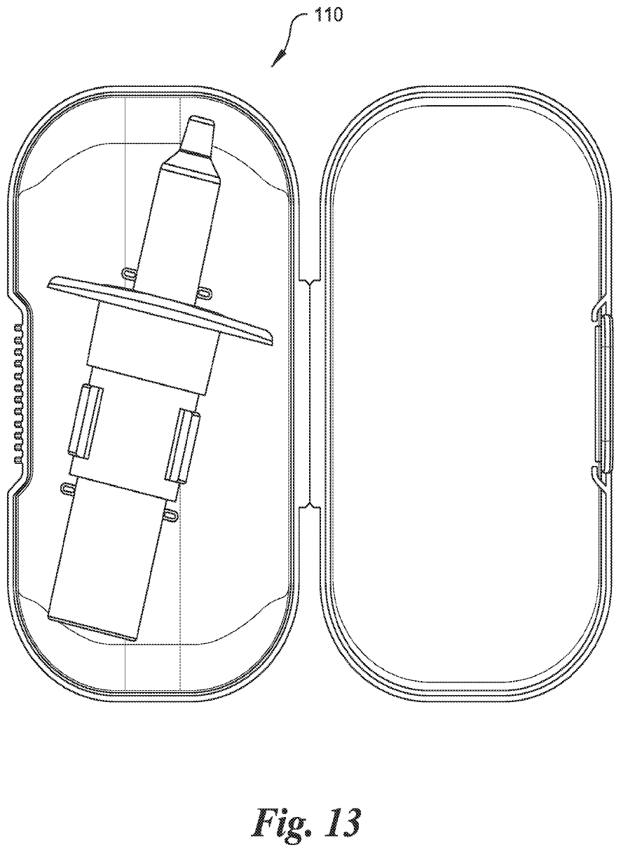

Optionally, as shown in FIGS. 13 and 14, when the device 112 can be placed within the clip 152, a longitudinal axis A_{D} of the device 112 extends at an angle θ that is not parallel or perpendicular to the longitudinal axis A_{L} of the secondary packaging 110 or the hinge axis A_{H}. The angle θ can optionally be from 10-80 degrees, optionally from 11-20 degrees, optionally from 12-16 degrees, optionally from 12.5-15 degrees, optionally 13.3 degrees, optionally from 20-70 degrees, optionally 18-22 degrees, optionally 20 degrees, optionally from 30-60 degrees, optionally 28-32 degrees, optionally 30 degrees, optionally 40-50 degrees, or optionally 45 degrees. The angle θ reduces the likelihood of damage to the device 112 if and/or when the secondary packaging 110 experiences a sudden force, such as upon dropping the secondary packaging 110. In particular, the angle θ changes the vector of the drop force(s), which makes the device 112 less likely to slip and/or move as rapidly as compared to if the longitudinal axis A_{D} of the device 112 is parallel to the direction of drop.

In another optional embodiment, the longitudinal axis A_{D} of the device 112 extends parallel to the longitudinal axis A_{L} of the secondary packaging 110 or the hinge axis A_{H}.

Another distinguishing feature of one optional embodiment of the secondary packaging 110 of the present embodiment is that the body 114 is designed and/or configured such that the clip 152 can only receive or hold the device 112 when the device 112 is pointing in one direction or in one orientation. For example. the secondary packaging 110 can optionally receive and hold the device 112 when the device 112 is oriented in the first direction, but not receive or hold the device 112 when the device is oriented in the opposing second direction.

To dictate or at least contribute to the functionality described above, as shown in FIGS. 13-15, at least two or four or more spaced-apart prongs 136 can extend inwardly and/or upwardly from the interior surface of the body 114. Thus, each prong 136 can extend inwardly into the cavity 116 when the secondary packaging 110 is in a closed configuration. Each prong 136 is spaced-apart from the clip 152 along the longitudinal axis A_{D} of the device 112.

Optionally, each prong 136 extends in parallel to any other prong 136. Each prong 136 can be wider than it is thick, and taller than it is wide. A base of each prong 136, which is attached to the interior surface of the body 114, can be bigger or wider than a free end of each prong 136. A tapered or concave portion can be located between the base and the free end of each prong 136. The tapered or concave portion of each prong 136 can face the device 112 when the device is positioned in the clip 152. Thus, each tapered or concave portion can accommodate or complement at least a portion of the device 112 when the device 112 is held by the clip 152.

In one optional embodiment, two of the four spaced-apart prongs 136 are on a first side of the clip 152 along the longitudinal axis A_{D} of the device 112, and two of the four spaced-apart prongs 136 are located on an opposing or second side of the clip 152 along the longitudinal axis A_{D} of the device 112.

Optionally, the prongs 136 on the first side are positioned closer to each other and/or closer to the longitudinal axis A_{D} of the device 112 than the prongs 136 on the second side. Stated differently, a first pair of the prongs 136 across the longitudinal axis A_{D} of the device 112 from each other can be closer together than a second pair of the prongs 136 across the longitudinal axis AD of the device 112 from each other.

Such an arrangement allows the device 112 to be attached to the clip 152 when the device is pointed in a first direction (e.g., with the nose of the device 112 between the closer pair of prongs 136), but prevents the device 112 from being attached to the clip 152 when the device is pointed in an opposing second direction (e.g., if a user were to attempt to insert the handle end of the device 112 between the closer pair of prongs 136).

In one optional embodiment, the clip 152 and prongs 136 can combine to prevent or at least limit movement of the device 112 at least in a direction perpendicular to the longitudinal axis A_{D} of the device 112 when the device 112 is attached to the clip 152 and/or the body 114. Further, portions of the prongs 136 can contact portions of the device 112 to prevent the device from moving in a direction parallel to the longitudinal axis A_{D} of the device 112 when the device 112 is attached to the clip 152 and/or the body 114. For example, one or more of the prongs 136 can abut an outer surface or ledge of the device 112 to prevent certain movement of the device 112, such as in a direction parallel to the longitudinal axis A_{D} of the device 112.

Referring to FIGS. 9, 11, and 13-15, in one optional embodiment, the secondary packaging 110 can include a locking, latching, or closure mechanism. More particularly, the cap 126 can include a latch 138 extending at least partially therefrom. The latch 138 can be configured to engage at least a portion of the body 114, such as at least one projection or a plurality of spaced-apart projections 140 extending from an exterior surface of the body 114, to help maintain the secondary packaging 110 in a closed configuration. Optionally, the plurality of spaced-apart projections 140 can extend in parallel at or near a lateral mid-portion of the body 114.

Optionally, the latch 138 can be generally flat or planar with a groove therein or an opening 142 extending therethrough. Each projection 140 can optionally be sized, shaped, and or configured to be at least slightly larger than the opening 142, such that the latch 138 can be snap-fit onto the projection(s) 140. In the same or in an alternative arrangement, one or both ends of each projection 140 can include a catch that can extend through and/or around the opening 142 and engage a front surface of the latch 138 when the secondary packaging 110 is in the closed configuration.

Engagement or contact between the latch 138 and the at least one projection 140 can maintain the secondary packaging 110 in the closed position. Optionally, an outer periphery of the opening 142 can engage the projection(s) 140 when the secondary packaging 110 is in the closed configuration. Separation of the latch 138 from the at least one projection 140 is required, in at least one optional embodiment, in order to reconfigure the secondary packaging 110 from the closed position to the open position.

Optionally, a method of attaching one or more of the devices 112 to the secondary packaging 110 can include pivoting the cap 126 with respect to the body 114 about the hinge 151 to move the secondary packaging 110 from the closed position to the open position to expose the cavity 116. Next, the device 112 can be placed, inserted, and/or snap-fit into the clip 152. In one optional embodiment, the device 112 must be oriented in a particular manner so as to be received by at least a pair of the prongs 136. Otherwise, the device 112 may not be able to contact and engage the clip 152. Optionally, prior to pivoting the cap 126 with respect to the body 114, the latch 138 is separated from the at least one projection 140.

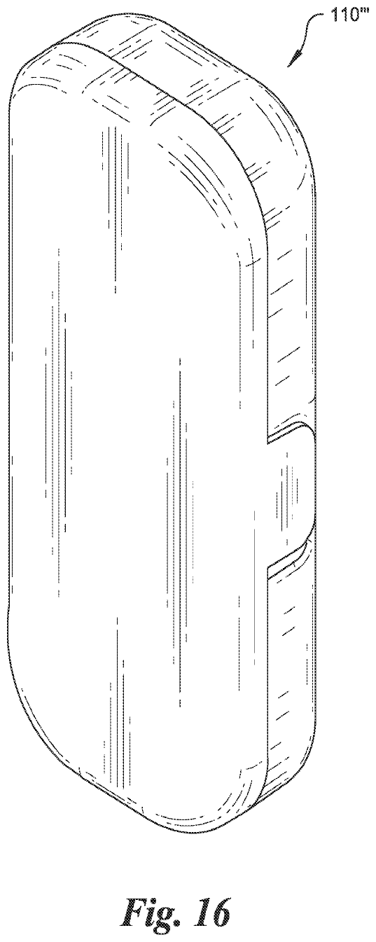

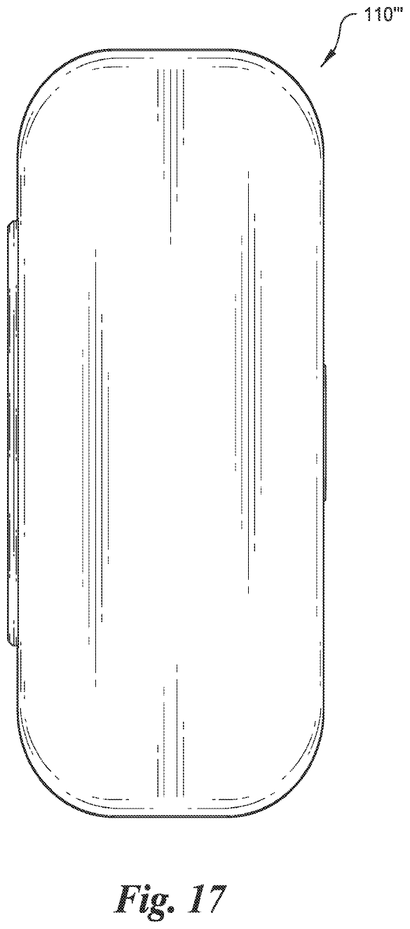

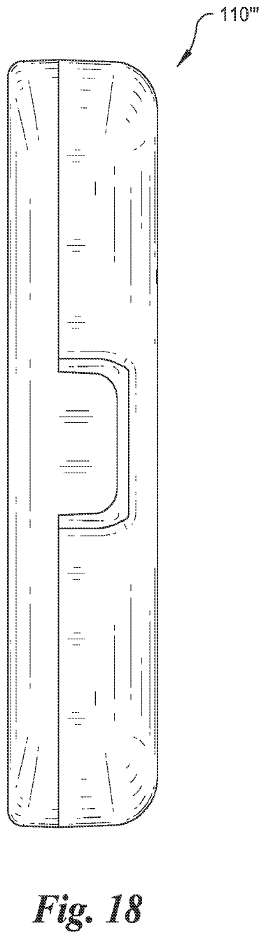

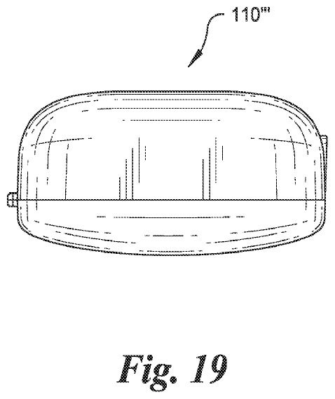

FIGS. 16-20 show yet another alternative optional embodiment of the combination of the presently disclosed technology. The secondary packaging 110″′ shown in FIGS. 16-20 has numerous components or aspects that are similar to the combination shown in FIGS. 9-15 and discussed above. As such, similar or identical structure as between the embodiment of FIGS. 9-15 and the embodiment of FIGS. 16-20 is distinguished in FIGS. 16-20 by a three prime symbols (′″). Description of certain similarities between the embodiment of FIGS. 9-15 and the embodiment of FIGS. 16-20 may be omitted herein for convenience and brevity only.

One distinguishing feature of the secondary packaging 110′″ of the optional embodiment shown in FIGS. 16-20 is the ability to hold and/or protect two devices 112 when the secondary packaging 110′″ is in a closed configuration. To accommodate the additional device 112, the secondary packaging 110′″ may be wider and/or longer, but not necessarily higher or thicker, than the secondary packaging 110 shown in FIGS. 9-15. The secondary packaging 110″′ can optionally include two spaced-apart clips 152″′.

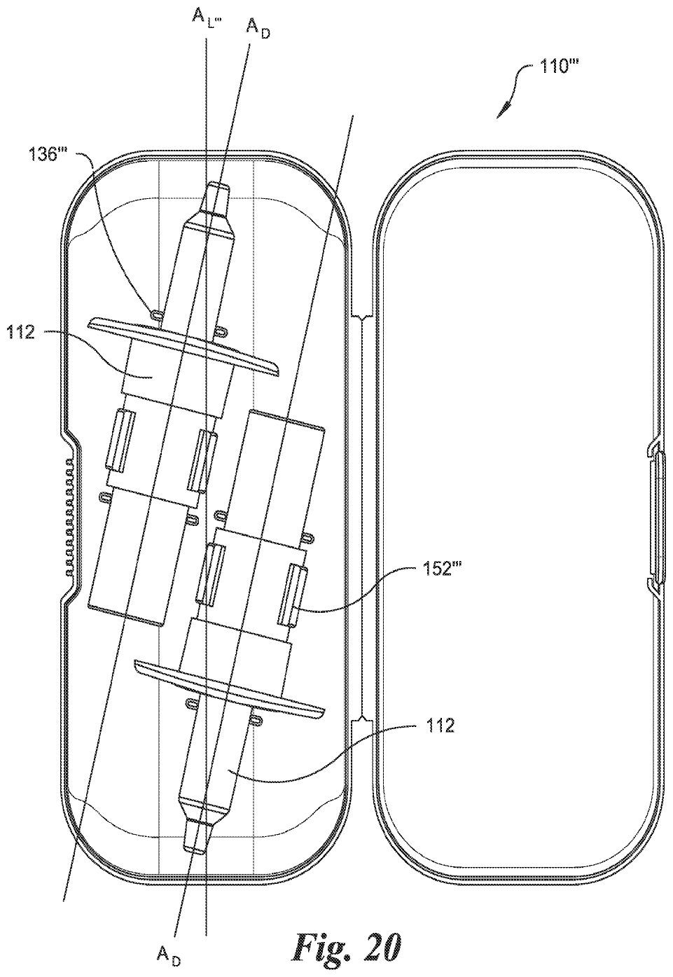

Referring to FIG. 20, the secondary packaging 110′″ can optionally be configured to receive one of the devices 112 in a first direction and receive the second of the devices 112 in an opposing second direction. A longitudinal axis A_{D} of each device 112 can extend at the angle θ that is not parallel or perpendicular to the longitudinal axis A_{L}″′ of the secondary packaging 110″′. To permit such an orientation, eight or more of the prongs 136′″ are arranged in a staggered manner.

The following exemplary embodiments further describe optional aspects of the presently disclosed technology and are part of this Detailed Description. These exemplary embodiments are set forth in a format substantially akin to claims (each with numerical designations followed by a capital letter), although they are not technically claims of the present application. The following exemplary embodiments refer to each other in dependent relationships as “embodiments” instead of “claims.”

1A. A combination comprising:
a device for administering medicine or a drug; and
two piece secondary packaging configured to hold the device, the secondary packaging comprising a body and a cap separable therefrom, the body and cap combining to form a cavity configured to sandwich the device therein.

2A. The combination embodiment 1A, wherein the device is an inhaler.

3A. The combination according to embodiment 2A, wherein the inhaler is reconfigurable between a non-activated configuration wherein the inhaler does not release or administer the medicine or the drug and an activated configuration wherein the inhaler does release or administer the medicine or the drug.

4A. The combination according to embodiment 3A, wherein the secondary packaging is not moisture tight when the cap is attached to the body.

1B. A one-piece secondary packaging configured to hold an inhaler, the secondary packaging comprising:
a cap pivotably attached to a body by a hinge, each of the cap and the body including two spaced-apart ribs extending inwardly therefrom, at least one of the cap and the body including a clip on an interior surface thereof, the clip being configured to removable receive the inhaler.

2B. The secondary packaging according to embodiment 1B, wherein each rib includes a cut-out therein, each cut-out being sized and shaped to receive at least a portion of the inhaler.

3B. The secondary packaging according to embodiment 1B or 2B, wherein the clip includes at least two opposing arcuate arms.

4B. The secondary packaging according to embodiment 1B, 2B, or 3B, wherein the secondary packaging is configured to prevent the device from being reconfigured from the non-activated position to an activated position when the cap is in the closed position.

5B. The secondary packaging of any one of embodiments 1B-4B, wherein the inhaler is a nasal inhaler.

1C. Secondary packaging configured to hold a device for administering medicine or a drug, the secondary packaging comprising:
a body having a base and a sidewall extending upwardly therefrom; and
a cap removably attachable to the body,
wherein the cap and the body combine to form a cavity for holding the device when the cap is attached to the body.

2C. The secondary packaging of embodiment 1C, wherein, when the device is within the cavity and the cap is attached to the body, at least a portion of the body and the cap are configured to prevent movement of the device that would otherwise release or administer the medicine or the drug.

3C. The secondary packaging of embodiment 1C or 2C, wherein one end of the sidewall opposite the base of the body includes a rim that defines an opening leading to an interior of the body.

4C. The secondary packaging of any one of embodiment 1C-3C, wherein the cap includes a top wall and a sidewall extending downwardly therefrom, wherein the cap further includes an annular skirt extending downwardly from the sidewall.

5C. The secondary packaging of any one of embodiment 1C-4C, wherein at least a portion of the skirt is configured to fit within at least a portion of the sidewall of the body when the cap is attached to the body.

6C. The secondary packaging of any one of embodiment 1C-5C, wherein the entire skirt is configured to fit within at least a portion of the sidewall of the base when the cap is attached to the body.

7C. The secondary packaging of any one of embodiment 1C-6C, wherein the skirt includes at least one projection extending radially outwardly therefrom, and wherein the sidewall of the body includes at least one opening or groove therein, the at least one opening or groove of the sidewall of the body being configured to receive at least a portion of the at least one projection when the cap is attached to the body.

8C. The secondary packaging of any one of embodiment 1C-7C, wherein the skirt includes at least two spaced-apart cut-outs.

9C. The secondary packaging of any one of embodiment 1C-8C, wherein the cap includes two opposing cap thumb tabs, and wherein the body includes two opposing body thumb tabs.

10C. The secondary packaging of any one of embodiment 1C-9C, wherein simultaneously engaging at least two of the opposing cap or body thumb tabs causes the at least one projection to disengage from the at least one opening or groove.

11C. The secondary packaging of any one of embodiment 1C-10C, wherein a ring extends inwardly from an interior surface of the base of the body, the ring being configured to receive a portion of the device therein when the device is placed in the body.

12C. The secondary packaging of any one of embodiment 1C-11C, wherein the body includes at least two spaced-apart lower fins extending inwardly from an interior surface of the sidewall of the body, the lower fins combine to hold the device in a desired position within the secondary packaging.

13C. The secondary packaging of any one of embodiment 1C-12C, wherein the cap includes at least two spaced-apart upper fins extending inwardly from an interior surface of the sidewall of the cap, the upper fins combine to hold the device in a desired position within the secondary packaging.

14C. The secondary packaging of any one of embodiment 1C-13C, wherein the device is a nasal dose inhaler.

1D. A combination comprising:
a nasal dose inhaler; and
secondary packaging configured to hold the nasal dose inhaler, the secondary packaging comprising:
a body having a base and a sidewall extending upwardly therefrom; and
a cap removably attachable to the body,
wherein the cap and the body combine to form a cavity for holding the device when the cap is attached to the body.

1E. A method of protecting a device for administering a drug comprises providing secondary packaging that includes a clip attached to an interior of a body of the secondary packaging, the clip being configured to hold the device stationary with respect from the secondary packaging, a device axis of the device extending at an angle with respect to a hinge axis of the secondary packaging.

2E. The method of embodiment 1E, wherein then angle reduces the trauma or force experienced by the device upon impact on the secondary packaging.

While the presently disclosed technology has been described in detail and with reference to specific examples thereof, it will be apparent to one skilled in the art that various changes and modifications can be made therein without departing from the spirit and scope thereof. It is understood, therefore, that the presently disclosed technology is not limited to the particular embodiments disclosed, but it is intended to cover modifications within the spirit and scope of the presently disclosed technology.

1. Secondary packaging configured to hold at least one device for administering a drug, the secondary packaging comprising:
a body including at least one clip attached to an interior surface thereof, the at least one clip being configured to surround at least a portion of the at least one device, the clip being configured to prevent inadvertent removal or movement of the at least one device with respect to the body; and
a cap pivotably attached to the body, the cap and the body combine to form a cavity for holding the at least one device when the cap is in a closed position with respect to the body.

2. The secondary packaging of claim 1, wherein the at least one device is a nasal dose inhaler.

3. The secondary packaging of claim 1, wherein the body is configured such that the at least one clip can only receive the at least one device when the at least one device is pointing in one direction.

4. The secondary packaging of claim 3, wherein at least two spaced-apart prongs extend inwardly or upwardly from the interior surface of the body, the at least two spaced-apart prongs being spaced-apart from the at least one clip.

5. The secondary packaging of claim 4, wherein the at least two spaced-apart prongs comprise at least four spaced-apart prongs.

6. The secondary packaging of claim 5, wherein two of the at least four spaced-apart prongs are on a first side of a device axis that extends at an angle with respect to a hinge that attaches the body to the cover, and wherein two of the at least four spaced-apart prongs are on a second side of the device axis.

7. The secondary packaging of claim 1, wherein, when the at least one device is attached to the body, the body is configured to prevent movement of the at least one device that would otherwise release or administer the drug.

8. The secondary packaging of claim 1, wherein when the at least one device is attached to the body, the at least one device defines a device axis that extends at an angle with respect to a hinge axis, wherein the angle is from 10-80 degrees.

9. The secondary packaging of any previous claim 1, wherein the at least one clip includes two spaced-apart and arcuate arms.

10. The secondary packaging of claim 9, wherein each arcuate arm of the at least one clip includes a first portion that is concave with respect to the device axis and at least one second portion that is convex with respect to the device axis.

11. The secondary packaging of any claim 1, wherein the cap includes a latch extending at least partially therefrom, the latch being configured to engage at least one projection extending from an exterior surface of the body.

12. The secondary packaging of claim 11, wherein the latch includes an opening therein, an outer periphery of the opening being configured to engage the at least one projection when the secondary packaging is in the closed position.

13. The secondary packaging of claim 1, wherein the at least one device includes two devices, the two devices extending in opposite directions when attached to the body.

14. A method of attaching at least one device for administering a drug to secondary packaging, the method comprising:
pivoting a cap of the secondary packaging with respect to a body of the secondary packaging about a hinge of the secondary packaging to move the secondary packaging from a closed configuration to an open configuration to expose a cavity; and
placing the at least one device for administering the drug into at least one clip attached to an interior surface of the body of the secondary packaging, the at least one clip being configured to surround at least a portion of the at least one device, the clip being configured to prevent inadvertent removal or movement of the at least one device with respect to the body.

15. The method of claim 14, wherein a device axis of the at least one device extends at an angle with respect to a hinge axis of the secondary packaging.

16. The method of claim 14, wherein the angle is from 10-80 degrees.

17. (canceled)

18. The method of claim 14, wherein the body is configured such that the at least one clip can only receive the at least one device when the at least one device is pointing in one direction.

19. The method of claim 18, wherein at least two spaced-apart prongs extend inwardly or upwardly from the interior surface of the body, the at least two spaced-apart prongs being spaced-apart from the at least one clip.

20. (canceled)

21. (canceled)

22. (canceled)

23. (canceled)

24, (Canceled)

25. (canceled)

26. (canceled)

27. A method of treating a patient in need of a drug that is administered intranasally, the method comprising:
providing the secondary packaging of claim 1 with a drug delivery device stored therein, the drug delivery device comprising a body having a discharge end defining a drug discharge port and an opposed actuator end, the drug delivery device including a drug reservoir having a drug stored therein, the drug delivery device further including an actuator for ejecting the drug from the reservoir through the discharge port, the actuator extending out of the actuator end of the body, the actuator being linearly movable from an extended position to a depressed position, the discharge port being sealed when the actuator is in the extended position and the discharge port being open when the actuator is in the depressed position, the actuator being operable to discharge the drug from the reservoir upon axial movement of the actuator from the extended position to the depressed position;
removing the drug delivery device from the secondary packaging;
inserting the discharge end into a nostril of the patient; and
actuating the drug delivery device to intranasally administer the drug to the patient.

28. The method of claim 27, wherein the drug is a formulation comprising one or more of the following active pharmaceutical ingredients: Naloxone, Ketamine, Midazolam, Fentanyl, Lorazepam, Nalmefene, Epinephrine, Apomorphine, Cetrorelix, Loxapine, Eletriptan, Ketorolac, Glucagon and Olanzapine.
